(19) United States
(12) Patent Application Publication
(10) Pub. No.: US 2009/0028965 A1
(43) Pub. Date: Jan. 29, 2009

(54) MULTI-PART DISINFECTANT

(75) Inventor: Brian James Healey, East Harling (GB)

Correspondence Address: BARNES & THORNBURG LLP, P.O. BOX 2786, CHICAGO, IL, 60690-2786, US

(73) Assignee: Clinimax Limited, Bury St. Edmunds (GB)

(21) Appl. No.: 11/828,838

(22) Filed: Jul. 26, 2007

(51) Int. Cl. A61K 33/14; A61L 9/00

(52) U.S. Cl. 424/661; 422/29

(57) ABSTRACT

Disclosed is a multi-part disinfectant composition, wherein the parts thereof are solid and are packaged separately prior to use, and wherein the separately-packaged parts, when combined in the presence of water or an aqueous solution, react to form chlorine dioxide.

### FIELD OF THE INVENTION

This invention relates to a multi-component disinfectant, the components of which may be combined to form an active antimicrobial substance (especially chlorine dioxide), and a method of making and using a multi-component disinfectant.

### BACKGROUND OF THE INVENTION

Chlorine dioxide, ClO₂, is a gas which is freely soluble in water. It is widely used as a disinfectant, especially in aqueous systems e.g. rendering water potable, disinfecting cooling systems etc.

Chlorine dioxide gas is potentially explosive and cannot be stored for long periods in aqueous solution without degradation, so the substance is generally prepared in situ. It is known therefore to provide a dry powder comprising reagents which generate ClO₂ when added to water. However, these are not ideal as even mere traces of water are sufficient to bring about the reaction. As a result, either extreme precautions must be taken to prevent any moisture contacting the powder until desired, or else one must accept deterioration of the effectiveness of the powder over time. In addition, single part formulations are often very oxidising in their own right and hence highly corrosive. This can cause difficulties in retaining the composition in a container over prolonged periods.

The present invention aims to overcome or ameliorate these difficulties.

### SUMMARY OF THE INVENTION

In a first aspect, the invention provides a multi-part disinfectant composition, wherein the parts thereof are solid and packaged separately prior to use, and wherein the separately-packaged parts, when combined in the presence of water or an aqueous solution, react to form chlorine dioxide.

The term “multi-part”, as used herein, means that the composition comprises two or more separate parts. The disinfectant composition of the invention preferably comprises two separately-packaged parts, but there is no reason why the composition may not comprise three or more separately packaged parts.

The reader will readily appreciate that the parts of the composition may be combined in any order (e.g. one part may be added to water, and a second part added; or first and second parts may be simultaneously added to a desired volume of water).

Each part may comprise a single ingredient. Alternatively one or more of the parts of the composition may comprise two or more ingredients.

For simplicity, the composition will typically comprise two parts, and these may be referred to as, for example, Part 1 and Part 2, or Part A and Part B.

The separately-packaged parts of the composition may be stored for prolonged periods (e.g. over 1 year, typically over 2 years and even for 3 years or more) without significant diminution in efficacy, until desired. When the disinfectant is required the separate parts may be combined, in the presence of water or an aqueous solution, to produce ClO₂ in amounts substantially identical to the amounts which would have been produced had the parts been combined without prior storage. (For present purposes, “substantially identical” amounts of ClO₂ means at least 90%, preferably at least 95%, and most preferably at least 98% of the amount of ClO₂ produced when the parts are combined without prior storage.)

In one embodiment one of the parts of the disinfectant comprises a water-soluble chlorite, preferably an alkali metal chlorite or alkaline metal chlorite. In one embodiment one of the parts of the disinfectant comprises a mono-, di- or tri-chloroisocyanurate. In a preferred embodiment, the composition comprises two parts, one of which comprises an alkali metal chlorite or alkaline metal chlorite, and the other part comprises a mono-, di- or tri-chloroisocyanurate.

In a preferred embodiment, one part of the composition comprises sodium chlorite. In a preferred embodiment one part of the composition comprises dichloro- or trichloroisocyanurate. A particularly preferred ingredient is sodium dichloroisocyanurate. Chloroisocyanurate is readily available commercially. It is used to disinfect swimming pools, by generating hypochlorous acid in aqua.

Without wishing to be bound by any particular theory, the inventor believes that the chlorite and chloroisocyanurate react, in the presence of water in the following manner:
(i) dissolving the dichloroisocyanurate in water produces sodium hypochlorite and hypochlorous acid;

C₃Cl₂N₃O₃Na+2H₂O→C₃H₃N₃O₃+NaOCl+HOCl
(ii) the sodium chlorite reacts with hypochlorite, in acidic conditions, to produce chlorine dioxide:

H⁺+NaOCl+2NaClO₂→2ClO₂+Na⁺+NaOH+NaCl

The inventor has found that acidic conditions are required for optimal ClO₂ generation. Accordingly, it is preferred that the composition comprises (in one or more parts thereof) an acidic substance to lower the pH below 7. Preferably the composition is formulated such that, when mixed with the appropriate volume of tap water or aqueous solution, it produces an aqueous mixture with a pH in the range 5-6.5, preferably 5-6, most preferably about 5.5.

The composition preferably comprises a buffer and/or a sequestering agent. The sequestering agent, if present, should preferably be suitable for use at a pH in the range 5-6.5 and should be capable, in particular, of complexing calcium ions at those pH values. A suitable substance includes di-sodium hydrogen orthophosphate or hexametaphosphate.

In a preferred embodiment the disinfectant, once prepared, is active against Mycobacteria, especially Mycobacterium tuberculosis, the causative agent of tuberculosis. “Active”, in this sense, means that the prepared disinfectant at a dilution of 1 in 80 passes the test criteria of British Standard BS 6734:2004 when tested against Mycobacterium fortuitum NCTC 8573 with a one hour exposure time. Prepared disinfectants, in accordance with present invention, have been found to be extremely effective against mycobacteria and also spores of clostridium, such as C. difficile.

All Mycobacterial cells are surrounded by a relatively thick, waxy lipid coat. To enable the dissolved ClO₂ to attack the bacterial envelope it is necessary to remove the lipid coat. Preferably, the disinfectant composition of the invention comprises an ingredient which can achieve this. Conveniently, therefore, one or more parts of the disinfectant will comprise a detergent. The detergent is preferably readily water-soluble. The detergent is preferably one which is not susceptible to the oxidising effects of ClO₂ (e.g. ethoxylate-based detergents are not appropriate). A preferred detergent comprises sodium lauryl sulphate (SLS). As well as facilitating penetration of bacterial cells and spores, the detergent aids wetting by lowering surface tension.

A further preferred feature of the invention is the inclusion of a tableting aid to help compression and binding of the composition if a tablet form is desired. The tableting aid may be included in all parts of the composition which are to be tableted. Suitable tableting aids and/or binders include kaolin.

An additional preferred ingredient of the composition of the invention is a dissolution aid, which is especially useful if the formulation is provided wholly or partly in tablet form. One suitable dissolution aid comprises sodium carbonate, which effervesces in aqueous acidic conditions.

A typical formulation would comprise, split among two or more parts, the following amounts (in grams per 100 gms of formulation):

| | Typical | Preferred |
|---|---|---|
| Substance | Range | Amount |
| Citric Acid | 25-40 | 33.5 |
| (Sodium) Chlorite | 18-28 | 24 |
| (Sodium) dichloroisocyanurate | 12-22 | 16.5 |
| Disodium Hydrogen Phosphate | 10-20 | 13 |
| Sodium Carbonate | 5-10 | 5 |
| Detergent (e.g. SLS) | 4-10 | 8 |

Preferably the chlorite and the dichloroisocyanurate are in separate parts of the composition. In one embodiment the dichloroisocyanurate is packaged, together with the citric acid, in one part, and the remaining ingredients are packaged together in a second part.

If desired one or more parts may comprise a bulking agent or filler. Conveniently, one or more parts of the composition may be packaged together with a desiccant, such as silica gel.

In one embodiment, each part of the composition is packaged in a sealed container or package, but the separate sealed containers or packages may themselves be supplied in a single larger container or package which comprises all the parts of the composition needed to make a disinfectant when added to water or an aqueous solution.

In a second aspect, the invention provides a method of making a multi-part disinfectant composition in accordance with the first aspect of the invention, the method comprising the step of packaging, separately, two or more parts of the composition which parts, when combined in the presence of water or an aqueous solution, react to form an active antimicrobial substance, especially chlorine dioxide.

In a third aspect the invention provides a method of forming a solution containing an active antimicrobial substance, the method comprising the steps of combining, in the presence of water or an aqueous solution, the parts of a multi-part composition in accordance with the first aspect of the invention.

In a fourth aspect the invention provides a method of killing bacteria and bacterial spores, in particular Mycobacterium tuberculosis and/or Clostridium difficile or spores thereof, the method comprising the steps of: preparing an antibacterial liquid by combining in water or aqueous solution the parts of a multiple-part disinfectant composition according to claim 1; and exposing the bacteria or spores to an effective amount of the antibacterial liquid for an appropriate period of time.

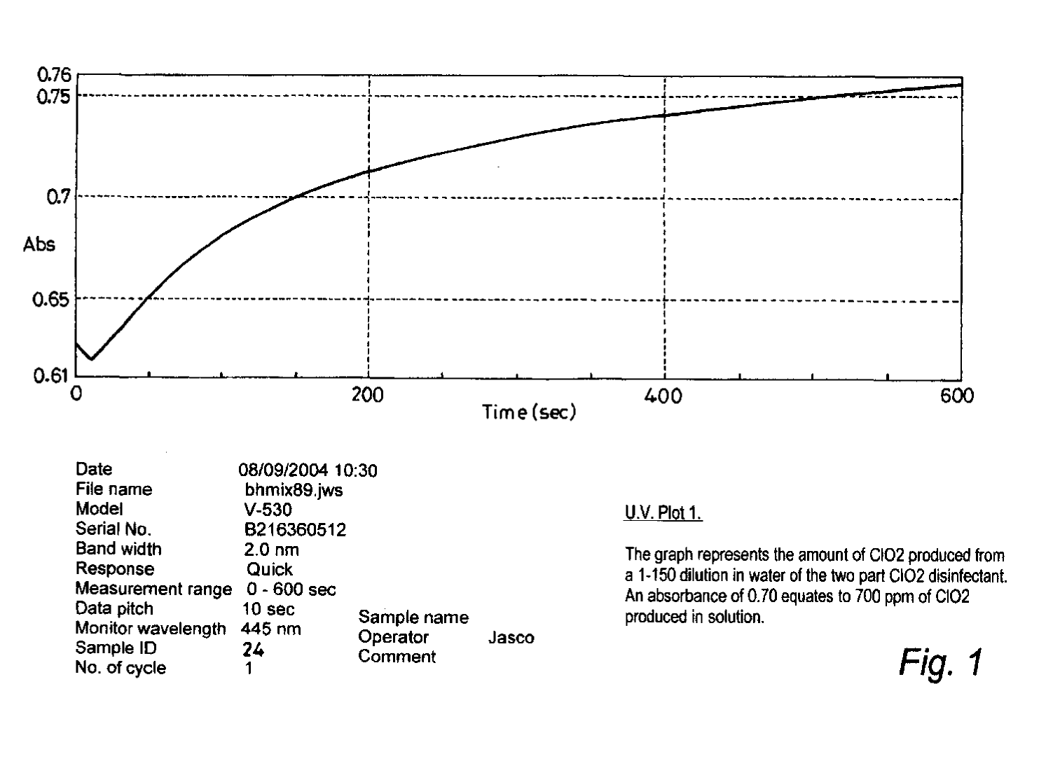

The invention will now be further described by way of illustrative examples and with reference to the accompanying drawing, FIG. 1, which is a graph of Absorbance against time, showing the amount of ClO₂ produced upon dissolving a multi-part composition according to the invention in water.

### EXAMPLE 1

A Two-Part Chlorine Dioxide producing Powder Disinfectant was prepared as follows:

Part A

| | |
|---|---|
| Sodium Chlorite | 45.4 g |
| Disodium Hydrogen Phosphate | 30.3 g |
| Sodium Carbonate | 15.1 g |
| Sodium Lauryl Sulphate | 9.2 g |
| | 100 g |

Part B

| | |
|---|---|
| Citric Acid | 66.7 g |
| Sodium Dichloroisocyanurate | 33.3 g |
| | 100 g |

An aqueous solution was prepared from the two parts above as follows:

| | |
|---|---|
| Part A: | 0.33 g |
| Part B: | 0.33 g |
| Water: | 99.34 g |

This equates to a 1:150 dilution. The Chlorine Dioxide produced in solution was measured by U.V. spectroscopy at 10 second intervals over a period of 10 minutes after preparation. Results are shown in FIG. 1. An absorbance of 0.70 equates to a concentration of dissolved ClO₂ of 700 ppm. The 1:150 dilution gave a total release after 10 minutes of 756 ppm of chlorine dioxide.

### EXAMPLE 2

A Two Part powder disinfectant prepared according to Example 1 was stored in Greiner Bio one Sample pots at ambient temperature for 250 days. The release of Chlorine Dioxide after the 250 days of a 1:150 dilution in water was 790 ppm after 10 minutes testing.

A single part Chlorine Dioxide releasing Powder Disinfectant stored under the same conditions for the same time released less than 100 ppm of Chlorine Dioxide after 10 minutes on the same test.

### EXAMPLE 3

A preparation according to Example 1 and 2 was prepared in a fresh aqueous solution at a dilution of 1:100 and was bacteriologically tested against several bacterial strains, according to BS EN 1276*. British Standards Institute

Strains tested were:

| | |
|---|---|
| MR⁺ Staphylococcus aureus | NCTC 12493 |
| MR⁺ Staphylococcus aureus | ATCC 33591 |
| Escherichia coli | ATCC 10536 |
| Enterococcus hirae | ATCC 10541 |
| Salmonella typimurium | NCTC 5710 |
| Salmonella enteridius | NCTC 4444 |

(⁺Methicillin-resistant)

In all cases a log reduction in excess of five fold was achieved down to less than 10 organisms per ml.

### EXAMPLE 4

A preparation according to Example 1 and 2 was prepared in a fresh aqueous solution at a dilution of 1:80 and was bacteriologically tested against:

| | |
|---|---|
| Mycobacterium fortutium | NCTC 8573 |

This was tested using the BS 6734:2004* test procedure with a 1 hour exposure time and passed the test criteria at the 1:80 dilution.

### EXAMPLE 5

A Two-Part Chlorine Dioxide producing Powder Disinfectant was prepared as follows:

Part A

| | |
|---|---|
| Sodium Chlorite | 48.4 g |
| Disodium Hydrogen Phosphate | 26.0 g |
| Sodium Carbonate | 9.6 g |
| Sodium Lauryl Sulphate | 16.0 g |
| | 100 g |

Part B

| | |
|---|---|
| Citric Acid | 66.7 g |
| Sodium Dichloroisocyanurate | 33.3 g |
| | 100 g |

An aqueous solution was prepared from the two parts above as follows:

| | |
|---|---|
| Part A: | 0.33 g |
| Part B: | 0.33 g |
| Water: | 99.34 g |

This equates to a 1:150 dilution. The chlorine dioxide produced in solution was measured by U.V. spectroscopy at 10 second intervals over a period of 10 minutes after preparation.

### EXAMPLE 6

Perez et al., (American Journal of Infection Control, August 2005, p 320-325) prepared a liquid solution of chlorine dioxide containing 600 ppm. They reported that this solution gave a 6 log reduction in viable C. difficile spores after a 30 minute exposure.

The present applicants tested (according to British Standard BS EN1276: 1997) a formulation according to Example 5 above. At a dilution of 1 in 438, giving a dissolved chlorine dioxide level of 300 ppm, a similar 6 log reduction in viable spores of C. difficile was obtained after just 5 minutes' exposure. The tests conducted were broadly similar, so the difference in speed of killing of the spores (at half the concentration of dissolved chlorine dioxide) is phenomenal. Without wishing to be bound by any particular theory, the Applicants believe the difference may be due to the inclusion of a detergent, at relatively high levels, in the composition of the invention.

Further, it is believed that sodium carbonate and disodium hydrogen phosphate form a buffer system, maintaining a pH of about 5.0. Disodium hydrogen phosphate additionally acts as a water softener, thereby preventing or reducing the conversion of sodium lauryl sulphate to calcium or magnesium lauryl sulphate, which would otherwise reduce detergency.

1. A multi-part disinfectant composition, wherein the parts thereof are solid and are packaged separately prior to use, and wherein the separately-packaged parts, when combined in the presence of water or an aqueous solution, react to form chlorine dioxide.

2. A composition according to claim 1, wherein one of the parts comprises a water-soluble chlorite.

3. A composition according to claim 2, wherein the water-soluble chlorite is an alkali metal chlorite or an alkaline metal chlorite.

4. A composition according to claim 1, wherein one of the parts comprises a mono-, di- or tri-chloroisocyanurate.

5. A composition according to claim 1, wherein the composition comprises two parts, one of which comprises an alkali metal chlorite or an alkaline metal chlorite, and the other part comprises a mono-, di- or trichloroisocyanurate.

6. A composition according to claim 1, wherein one part of the composition comprises sodium chlorite.

7. A composition according to claim 1, wherein one part of the composition comprises di- or trichloroisocyanurate.

8. A composition according to claim 1, wherein one part of the composition comprises sodium dichloroisocyanurate.

9. A composition according to claim 1 formulated such that, when mixed with an appropriate volume of tap water or aqueous solution, the composition produces an aqueous mixture with a pH in the range 5-6.5, preferably 5-6.

10. A composition according to claim 1, further comprising a buffer and/or sequestering agent.

11. A composition according to claim 10, comprising a sequestering agent capable of complexing calcium ions at a pH in the range 5-6.5.

12. A composition according to claim 10, wherein the sequestering agent is di-sodium hydrogen orthophosphate or hexametaphosphate.

13. A composition according to claim 1, wherein one or more parts of the composition comprise a detergent.

14. A composition according to claim 13, wherein the detergent is sodium lauryl sulphate.

15. A composition according claim 1, comprising a tableting aid to help compression and binding of the composition.

16. A composition according to claim 15, wherein the tableting aid comprises kaolin.

17. A composition according to claim 1, wherein one or more parts are provided in tablet form, and the tableted part(s) comprises a dissolution aid.

18. A composition according to claim 17, wherein the dissolution aid comprises sodium carbonate.

19. A composition according to claim 1, comprising the following amounts of ingredients (in grams per 100 gms of formulation) split among two or more parts:

| | Typical | Preferred |
|---|---|---|
| Substance | Range | Amount |
| Citric acid | 25-40 | 33.5 |
| (Sodium) chlorite | 18-28 | 24 |
| (Sodium) dichloroisocyanurate | 12-22 | 16.5 |
| Disodium hydrogen phosphate | 10-20 | 13.0 |
| Sodium carbonate | 5-10 | 5.0 |
| Detergent | 4-10 | 8 |

20. A method of making a multi-part disinfectant composition according to claim 1, the method comprising the step of packaging, separately, two or more parts of the composition which parts, when combined in the presence of water or an aqueous solution, react to form chlorine dioxide.

21. A method of forming a solution containing an active antimicrobial substance, the method comprising the steps of combining, in the presence of water or an aqueous solution, the parts of a multi-part composition in accordance claim 1.

22. A method of killing bacteria and bacterial spores, in particular Mycobacterium tuberculosis and/or Clostridium difficile or spores thereof, the method comprising the steps of: preparing an antibacterial liquid by combining in water or aqueous solution the parts of a multi-part disinfectant composition according to claim 1; and exposing the bacteria or spores to an effective amount of the antibacterial liquid for an appropriate period of time.
